(19) United States
(12) Patent Application Publication
(10) Pub. No.: US 2005/0019425 A1
(43) Pub. Date: Jan. 27, 2005

(54) Composition and method

(76) Inventors: Shiguang Yu, Topeka, KS (US); Raymond Nachreiner, East Lansing, MI (US); Claudia Ann Kirk, Lawrence, KS (US); Karen J. Wedekind, Meriden, KS (US)

Correspondence Address: COLGATE-PALMOLIVE COMPANY, 909 RIVER ROAD, P.O. BOX 1343, PISCATAWAY, NJ, 08855-1343, US

(21) Appl. No.: 09/683,003

(22) Filed: Nov. 7, 2001

(51) Int. Cl. A61K 33/04; A61K 31/198

(52) U.S. Cl. 424/702; 514/561

(57) ABSTRACT

A composition which comprises about 0.1 to about 4.5 mg selenium/kg of diet on a dry matter basis.

### BACKGROUND OF INVENTION

Selenium is well known as an essential component of a mammal's diet. Many functions have been associated with it. These include growth, reproduction, antioxidant, endocrine function, immune function, normal hair growth, muscle, and heart function. Selenium has been used in hair shampoos as an antidandruff agent, see U.S. Pat. Nos. 5,798,121 and 5,702,690 as the disulphide and sulphide. Topical application of selenium has been implicated in hair growth, see U.S. Pat. No. 5,629,002 wherein inorganic selenium compounds together with thiocyanic compounds are disclosed as improving quality and stimulating the growth of hair. At column 1, lines 28-44 of 002, various materials including selenium, are disclosed as: “can display a more or less stimulating influence on the growth of the hair. However, the effects achieved thereby, as a rule, are not significant and not reproducible in practice.”

We have now found a range of dietary selenium that stimulates hair growth of dogs and cats receiving a properly nutritious diet. Quantitative measures show hair growth rate is statistically greater within this range. Below and above the range, hair growth is slowed. Providing a steady positive growth rate for hair can be desirable for esthetic benefits. Enhancing hair growth with selenium supplementation can benefit the management of disorders of the hair follicles and/or hair coat by preventing or treating poor hair growth or alopecia related to disease, reproductive cycle, seasonal changes or direct hair removal (e.g., shaving or clipping).

### SUMMARY OF INVENTION

In accordance with the invention, there is a composition which comprises about 0.1 to about 4.5 mg selenium/kg of diet on a dry matter basis, the diet providing nutritional sustenance. Generally, hair growth is slowed when dietary selenium concentration is below about 0.1 mg selenium/kg diet and above about 4.5 mg selenium/kg diet on a dry matter basis. Thus, the range between 0.1 and 4.5 mg selenium/kg diet maximizes hair growth.

A further aspect of the invention is a method for controlling the rate of hair growth in a dog or cat which comprises feeding the dog or cat a hair growth rate controlling amount of selenium of from about 0.1 to about 4.5 mg per kg diet on dry matter basis, the diet providing nutritional sustenance. Otherwise, the selenium can be fed the dog or cat as a supplement to the regular diet as long as the appropriate levels of selenium are maintained.

### DETAILED DESCRIPTION

Selenium can be administered orally to the dog or cat in any form as long as it is absorbed by the animals. Examples of such forms of selenium include salts such as selenites and selenates, for example, sodium selenite, and sodium selenate; acids such as selenious and selenic acid; organic compounds containing selenium such as selenomethionine, selenocysteine, selenohomocystine, selenotaurine; and selenium containing food or ingredients such as meat, fish, and the like.

The quantity of selenium as disclosed in this specification is measured as the quantity of selenium per se. For example, if selenium is administered as selenomethionine, it is the actual quantity of selenium that is in the selenomethionine that is intended as the quantity disclosed. The minimum quantity of selenium that brings about the controlled rate of hair growth in cats and dogs is about 0.1 mg as measured per kg diet on dry matter basis. The maximum quantity is a controlling hair growth rate but non-toxic quantity of selenium. Generally, this is no more than about 4.5 mg, desirably no more than about 2 mg per kg diet on dry matter basis. A desirable minimum is about 0.5 mg per kg diet on dry matter basis. As stated previously, it has now been found that the control of hair growth rate through usage of selenium can result in a pronounced statistically significant hair growth acceleration depending upon the quantity of selenium employed. Hair growth rate increases and reaches its maximum when dietary selenium concentration is above 0.1 mg selenium/kg diet and hair growth is slowed when dietary selenium concentration is higher than about 4.5 mg selenium/kg. Selenium can be administered to the dog or cat in diet or through specific supplements to be fed to the animal.

Animals that benefit from increased hair growth rate are dogs, cats, ferrets, sheep, and other mammals.

In addition to optimizing growth of hair coat for esthetic benefits, enhancing hair growth with selenium supplementation can benefit the management of disorders of the skin, hair follicle and/or hair coat by preventing or treating poor hair growth or alopecia related to disease, reproductive cycle, seasonal changes or direct hair removal (e.g., shaving or clipping).

Below is an example of the invention showing the benefit and effect of using selenium.

### EXAMPLE 1

Adult beagles of both male and females are used. They are between 1.2 and 3.7 years of age at the start of the experiment.

Study Design: Thirty-six adult dogs are divided into six groups of six dogs in each group based on their age, gender and body condition score so that the averages of those three criteria are similar among groups. All dogs are given the basal diet for three weeks (pre-treatment period) before they are allocated to one of the experimental diets. The third week of the pre-treatment period is considered as week 0 of the study. Hair growth is measured in weeks 0, 11 and 22 of the study using the method described below. Dogs are fed with the experimental diets for 24 weeks.

A torula yeast basal diet is used. The basal diet is nutritionally complete and balanced for an adult dog except for selenium. Different amounts of selenium (as selenomethionine) are added into the basal diet to form six experimental diets which contain 0, 0.05, 0.1, 0.5, 1 or 5 mg selenium/kg diet dry matter, respectively. Selenium concentrations in the experimental diets are confirmed by chemical analyses. The analyzed dietary selenium concentrations were 0.034, 0.085, 0.123, 0.527, 1.025 and 5.045 mg/kg dry matter, respectively.

Hair Growth Measurement: Hairs in an area of 2×2 on the left side of the body above the hind leg and about 3 cm away from the spinal are clipped with an electronic clipper (Oster Pro™ Model 78400-01A, blade size: 40.1 mm, Oster Elite™). The hair is cut as close to the skin as possible. A tattoo dot of about 1 mm in diameter is made in the center of the clipped area using a tattoo marker (Spaulding Special Electric Tattoo Marker, Model SSEMK). Hair growth is measured by taking photographs from the same clipped area twice using the tattoo dot as a marker. The first photograph is taken right after the clipping and the second photograph four days later. A Polaroid close-up camera (CU-5) and Polaroid 665 films are used to take photographs. A glass slide is attached to the front frame of the camera to press hairs to the skin to minimize errors caused by the angle formed between standing hair and the skin. The image is magnified three times by the camera. Same exposure conditions are used for all dogs in the study. On each day of photograph shooting, a photograph is also taken of a ruler with clear markers under the same conditions and serves as a reference. After exposure, both prints and negatives of the films are treated according to the use directions. The negative films provide sharp and clear images of hairs and are used for hair growth measurement. The negative films are scanned (EPSON Expression 836XL) into a computer. Afterwards, the length of primary hairs in the film is measured using an image analyzing software (BioScan Optimas). The difference of the average hair lengths between the first and the second photographs from the same dog is the hair growth during the four-day period.

Results: There is no difference in hair growth among groups in week 0 (Table 1). However, dogs given diets containing 0.1, 0.5 or 1 mg selenium/kg diet have a significantly (p<0.05) higher rate of hair growth than dogs receiving diets with 0, 0.05 or 5 mg selenium/kg diet in week 11 and 22 of the study.

TABLE 1
Hair growth of Beagles fed diets with various levels of selenium

| | Hair growth (mm/day) | | |
|---|---|---|---|
| Dietary Se (mg/kg) | Week 0 | Week 11 | Week 22 |
| 0.034 | 0.21 | 0.12 | 0.12 |
| 0.085 | 0.18 | 0.15 | 0.11 |
| 0.123 | 0.13 | 0.26 | 0.20 |
| 0.527 | 0.15 | 0.22 | 0.23 |
| 1.025 | 0.18 | 0.26 | 0.20 |
| 5.045 | 0.20 | 0.14 | 0.13 |

No apparent clinical signs of selenium deficiency or toxicity are observed in the dogs during the study period. Average food intake and body weights are not affected by the dietary treatments. Blood CBC and chemistry measurements as well as serum total thyroxine and 3, 5, 3′triiodothyronine are within the normal ranges. These results demonstrate that dietary selenium concentrations from 0.1 to about 4.5 mg selenium/kg diet maximize hair growth of dogs, with preferred levels between about 0.1 and about 2 mg selenium/kg diet and most preferred at about 0.1 to about 1 mg selenium/kg diet dry matter.

1. A composition which comprises about 0.1 to about 4.5 mg selenium/kg of dog or cat diet on a dry matter basis, the diet providing nutritional sustenance for an animal.

2. A method for controlling the rate of hair growth in a dog or cat which comprises feeding the dog or cat a hair growth rate controlling amount of selenium of from about 0.1 to about 4.5 selenium mg /kg of diet on a dry matter basis.

3. A method for preventing or treating poor hair growth or alopecia in animals in need of said treatment, related to disease, reproduction cycle, seasonal changes or direct hair removal which comprises feeding the animal a hair growth rate controlling amount of selenium of from about 0.1 to about 4.5 mg/kg of diet on a dry matter basis.

4. The method in accordance with claim 3 wherein the animal is a dog, cat, or sheep.

5. The method in accordance with claim 4 wherein the animal is a dog or cat.
